(19) United States
(12) Patent Application Publication
(10) Pub. No.: US 2008/0262027 A1
(43) Pub. Date: Oct. 23, 2008

(54) Novel Chemical Compounds

(76) Inventors: Kevin J. Duffy, Collegeville, PA (US); Duke M. Fitch, Collegeville, PA (US); Antony N. Shaw, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/092,993

(22) PCT Filed: Nov. 3, 2006
(86) PCT No.: PCT/US06/60511

§ 371 Date: May 8, 2008

Related U.S. Application Data
(60) Provisional application No. 60/734,663, filed on Nov. 8, 2005.

(51) Int. Cl. A61K 31/437; A61P 7/06; A61P 7/00; C07D 471/04

(52) U.S. Cl. 514/300; 546/121

(57) ABSTRACT

This invention relates to newly identified compounds for inhibiting YAK3 proteins and methods for treating diseases associated with the imbalance or inappropriate activity of YAK3 proteins.

### FIELD OF THE INVENTION

This invention relates to newly identified compounds for inhibiting YAK3 proteins and methods for treating diseases associated with YAK3 activity.

### BACKGROUND OF THE INVENTION

A number of polypeptide growth factors and hormones mediate their cellular effects through a signal transduction pathway. Transduction of signals from the cell surface receptors for these ligands to intracellular effectors frequently involves phosphorylation or dephosphorylation of specific protein substrates by regulatory protein serine/threonine kinases (PSTK) and phosphatases. Serine/threonine phosphorylation is a major mediator of signal transduction in multicellular organisms. Receptor-bound, membrane-bound and intracellular PSTKs regulate cell proliferation, cell differentiation and signalling processes in many cell types.

Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are potential targets for drug design.

A subset of PSTKs are involved in regulation of cell cycling. These are the cyclin-dependent kinases or CDKs (Peter and Herskowitz, Cell 1994: 79, 181-184). CDKs are activated by binding to regulatory proteins called cyclins and control passage of the cell through specific cell cycle checkpoints. For example, CDK2 complexed with cyclin E allows cells to progress through the G1 to S phase transition. The complexes of CDKs and cyclins are subject to inhibition by low molecular weight proteins such as p 16 (Serrano et al, Nature 1993: 366, 704), which binds to and inhibits CDK4. Deletions or mutations in p 16 have been implicated in a variety of tumors (Kamb et al, Science 1994: 264, 436-440). Therefore, the proliferative state of cells and diseases associated with this state are dependent on the activity of CDKs and their associated regulatory molecules. In diseases such as cancer where inhibition of proliferation is desired, compounds that inhibit CDKs may be useful therapeutic agents. Conversely, activators of CDKs may be useful where enhancement of proliferation is needed, such as in the treatment of immunodeficiency.

YAK1, a PSTK with sequence homology to CDKs, was originally identified in yeast as a mediator of cell cycle arrest caused by inactivation of the cAMP-dependent protein kinase PKA (Garrett et al, Mol Cell Biol. 1991: 11-6045-4052). YAK1 kinase activity is low in cycling yeast but increases dramatically when the cells are arrested prior to the S-G2 transition. Increased expression of YAK1 causes growth arrest in yeast cells deficient in PKA. Therefore, YAK1 can act as a cell cycle suppressor in yeast.

U.S. Pat. No. 6,323,318 describes two novel human homologs of yeast YAK1 termed YAK3-2, one protein longer than the other by 20 amino acids. YAK3-2 proteins (otherwise reported as REDK-L and REDK-S in Blood, 1 May 2000, Vol 95, No. 9, pp 2838) are primarily localized in the nucleus. YAK-2 proteins (hereinafter simply referred as YAK3 or YAK3 proteins) are present in hematopoietic tissues, such as bone marrow and fetal liver, but the RNA is expressed at significant levels only in erythroid or erythropoietin (EPO)-responsive cells. Two forms of REDK cDNAs appear to be alternative splice products. Antisense REDK oligonucleotides promote erythroid colony formation by human bone marrow cells, without affecting colony-forming unit (CFU)-GM, CFU-G, or CFU-GEMM numbers. Maximal numbers of CFU-E and burst-forming unit-erythroid were increased, and CFU-E displayed increased sensitivity to suboptimal EPO concentrations. The data indicate that REDK acts as a brake to retard erythropoiesis. Thus inhibitors of YAK3 proteins are expected to stimulate proliferation of cells in which it is expressed. More particularly, inhibitors of YAK3 proteins are useful to treat or prevent diseases of the erythroid and hematopoietic systems associated with YAK3 activity, including but not limited to anemia, anemias due to renal insufficiency or to chronic disease, such as autoimmunity, HIV, or cancer, and drug-induced anemias, myelodysplastic syndrome, aplastic anemia and myelosuppression, and cytopenia.

### SUMMARY OF THE INVENTION

This invention relates to novel compounds of Formula I:

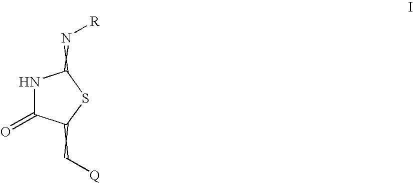

in which
R is selected from a group consisting of: aryl, substituted aryl, heteroaryl, substituted heteroaryl, alkyl, substituted alkyl, alkoxy, —N—R¹⁵, —O—R¹⁵, cycloalkyl, substituted cycloalkyl, cycloalkyl containing from 1 to 4 heteroatoms, substituted cycloalkyl containing from 1 to 4 heteroatoms,
   where R¹⁵ is C₁-C₁₂aryl, substituted C₁-C₁₂aryl, alkyl, substituted alkyl, cycloalkyl, substituted cycloalkyl, cycloalkyl containing from 1 to 4 heteroatoms, substituted cycloalkyl containing from 1 to 4 heteroatoms; and

### Q is

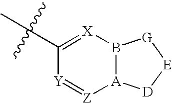

wherein,
A, B, D, E, and G together form a ring containing from 1 to 2 double bonds and from 1 to 4 nitrogens;
   where,
A and B are independently selected from a group consisting of: C and N;
G, E, and D are independently selected from a group consisting of: CR²⁰, O, S, and N;
X, Y and Z are CR²⁰;
where each R²⁰ is independently selected from the group consisting of: hydrogen, halogen, amino, alkylamine, substituted alkylamine, dialkylamine, substituted dialkylamine, hydroxy, alkoxy, alkyl, substituted alkyl, aryl, substituted aryl, heteroaryl, substituted heteroaryl, arylamine, substituted arylamine, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected from a group consisting of: hydrogen, C₁-C₄alkyl, and aryl, and R¹¹ and R¹² are independently selected from a group consisting of: hydrogen, C₁-C₄alkyl, and aryl;

provided that one and only one of A and B is N,

also provided that R is not unsubstituted phenyl;

and/or a pharmaceutically acceptable salt, hydrate, solvate or pro-drug thereof.

This invention relates a method of inhibiting YAK3 in a mammal; comprising, administering to the mammal a therapeutically effective amount of a compound of the formula I.

This invention relates to a method of treating or preventing diseases of the erythroid and hematopoietic systems, requiring YAK3 inhibition, including but not limited to, anemias due to renal insufficiency or to chronic disease, such as autoimmunity, HIV, or cancer, and drug-induced anemias, myelodysplastic syndrome, aplastic anemia and myelosuppression, and cytopenia; comprising administering to a mammal a therapeutically effective amount of a compound of formula I.

Included in the present invention are pharmaceutical compositions that comprise a pharmaceutical carrier and compounds useful in the methods of the invention.

Also included in the present invention are methods of co-administering the presently invented YAK3 inhibiting compounds with further active ingredients.

### DETAILED DESCRIPTION

This invention relates to compounds of Formula I as described above. Also included among the presently invented compounds of Formula I are those in which R is substituted aryl; and/or a pharmaceutically acceptable salt, hydrate, solvate or pro-drug thereof.

Included among the presently invented compounds of Formula I are those in which:
R is selected from a group consisting of: aryl, substituted aryl, heteroaryl, substituted heteroaryl, and

### Q is

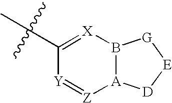

wherein,
A, B, D, E, and G together form a ring containing from 1 to 2 double bonds and from 1 to 2 nitrogens;
   where,
A and B are independently selected from a group consisting of: C and N;
G, E, and D are independently selected from a group consisting of: CR²⁰ and N;
X, Y and Z are CR²⁰;
where each R²⁰ is independently selected from the group consisting of: hydrogen, halogen, amino, alkylamine, substituted alkylamine, dialkylamine, substituted dialkylamine, hydroxy, alkoxy, alkyl, substituted alkyl, aryl, substituted aryl, heteroaryl, substituted heteroaryl, arylamine, substituted arylamine, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected from a group consisting of: hydrogen, C₁-C₄alkyl, and C₁-C₁₂aryl and R¹¹ and R¹² are independently selected from a group consisting of: hydrogen, C₁-C₄alkyl, and C₁-C₁₂aryl;

provided that one and only one of A and B is N,

also provided that R is not unsubstituted phenyl;

and/or a pharmaceutically acceptable salt, hydrate, solvate or pro-drug thereof.

Included among the presently invented compounds of Formula I are those in which:
R is selected from a group consisting of: substituted aryl and

### Q is

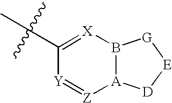

wherein,
A, B, D, E, and G together form a ring containing from 1 to 2 double bonds and from 1 to 2 nitrogens;
   where,
A and B are independently selected from a group consisting of: C and N;
G, E, and D are independently selected from a group consisting of: CR²⁰ and N;
X, Y and Z are CR²⁰;
where each R²⁰ is independently selected from the group consisting of: hydrogen, halogen, amino, alkylamine, substituted alkylamine, dialkylamine, substituted dialkylamine, hydroxy, alkoxy, alkyl, substituted alkyl, aryl, substituted aryl, heteroaryl, substituted heteroaryl, arylamine, substituted arylamine, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected from hydrogen, C₁-C₄alkyl, and C₁-C₁₂aryl, and R¹¹ and R¹² are independently selected from a group consisting of: hydrogen, C₁-C₄alkyl, and C₁-C₁₂aryl;

provided that one and only one of A and B is N;

and/or a pharmaceutically acceptable salt, hydrate, solvate or pro-drug thereof.

Included among the presently invented compounds of Formula I are those in which D is N; and/or a pharmaceutically acceptable salt, hydrate, solvate or pro-drug thereof.

Included among the presently invented compounds of Formula I are those in which:
R is C₁-C₁₂ substituted aryl, and

### Q is

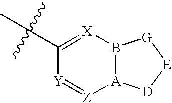

wherein,
A, B, D, E, and G together form a ring containing from 1 to 2 double bonds and from 1 to 2 nitrogens;
   where,
A and B are independently selected from a group consisting of: C and N;
G, and E are independently selected from a group consisting of CR²⁰ and N;
X, Y and Z are CR²⁰;
where each R²⁰ is independently selected from the group consisting of: hydrogen, halogen, amino, alkylamine, substituted alkylamine, dialkylamine, substituted dialkylamine, hydroxy, alkoxy, alkyl, substituted alkyl, aryl, substituted aryl, heteroaryl, substituted heteroaryl, arylamine, substituted arylamine, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected from a group consisting of: hydrogen, C₁-C₄alkyl, and C₁-C₁₂aryl, and R¹¹ and R¹² are independently selected from a group consisting of: hydrogen, C₁-C₄alkyl, and C₁-C₁₂aryl;

provided that one and only one of A and B is N,

and/or a pharmaceutically acceptable salt, hydrate, solvate or pro-drug thereof.

Included among the presently invented compounds of Formula I are those in which:
R is selected from a group consisting of: aryl, substituted aryl, heteroaryl, and substituted heteroaryl; and

### Q is

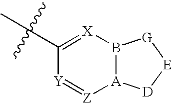

wherein,
A, B, D, E, and G together form a ring containing 2 double bonds and 2 nitrogens;
   where,
A and B are independently selected from a group consisting of: C and N;
G, and E are independently selected from a group consisting of: CR²⁰ and N;
X, Y and Z are CR²⁰;
where each R²⁰ is independently selected from the group consisting of: hydrogen, halogen, amino, alkylamine, substituted alkylamine, dialkylamine, substituted dialkylamine, hydroxy, alkoxy, alkyl, substituted alkyl, aryl, substituted aryl, heteroaryl, substituted heteroaryl, arylamine, substituted arylamine, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile, where, R¹⁰ is selected from a group consisting of: hydrogen, C₁-C₄alkyl, and
C₁-C₁₂aryl, and R¹¹ and R¹² are independently selected from a group consisting of: hydrogen, C₁-C₄alkyl, and C₁-C₁₂aryl;

provided that one and only one of A and B is N,

also provided that R is not unsubstituted phenyl;

and/or a pharmaceutically acceptable salt, hydrate, solvate or pro-drug thereof.

Included among the presently invented compounds of Formula I are those in which:
R is selected from a group consisting of: aryl, substituted aryl, heteroaryl, and substituted heteroaryl; and

### Q is

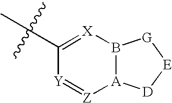

wherein,
A, B, D, E, and G together form a ring containing 2 double bonds and 2 nitrogens;
   where,
A and B are independently selected from a group consisting of: C and N;
G, and E are independently selected from a group consisting of: CR²⁰ and N;
D is N;
X, Y and Z are CR²⁰;
where each R²⁰ is independently selected from the group consisting of: hydrogen, halogen, amino, alkylamine, substituted alkylamine, dialkylamine, substituted dialkylamine, hydroxy, alkoxy, alkyl, substituted alkyl, aryl, substituted aryl, heteroaryl, substituted heteroaryl, arylamine, substituted arylamine, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile, where, R¹⁰ is selected from a group consisting of: hydrogen, C₁-C₄alkyl, and C₁-C₁₂aryl, and R¹¹ and R¹² are independently selected from a group consisting of: hydrogen, C₁-C₄alkyl, and C₁-C₁₂aryl;

provided that one and only one of A and B is N, also provided that R is not unsubstituted phenyl;

and/or a pharmaceutically acceptable salt, hydrate, solvate or pro-drug thereof.

Included among the presently invented compounds of Formula I are those in which:

R is substituted aryl;

### Q is

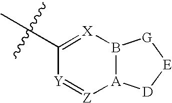

wherein,
A, B, D, E, and G together form a ring containing 2 double bonds and 2 nitrogens;
   where,
A and B are independently selected from a group consisting of: C and N;
G, and E are independently selected from a group consisting of: CR²⁰ and N;
X, Y and Z are CR²⁰;
where each R²⁰ is independently selected from the group consisting of: hydrogen, halogen, amino, alkylamine, substituted alkylamine, dialkylamine, substituted dialkylamine, hydroxy, alkoxy, alkyl, substituted alkyl, aryl, substituted aryl, heteroaryl, substituted heteroaryl, arylamine, substituted arylamine, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected from a group consisting of: hydrogen, C₁-C₄alkyl, and C₁-C₁₂aryl, and R¹¹ and R¹² are independently selected from a group consisting of: hydrogen, C₁-C₄alkyl, and C₁-C₁₂aryl;

provided that one and only one of A and B is N,

and/or a pharmaceutically acceptable salt, hydrate, solvate or pro-drug thereof.

Included among the presently invented compounds of Formula I are those in which:
R is C₁-C₁₂ substituted aryl, and
Q is

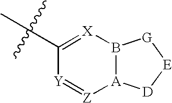

wherein,
A, B, D, E, and G together form a ring containing 2 double bonds and 2 nitrogens;
   where,
A and B are independently selected from a group consisting of: C and N;
G, and E are independently selected from a group consisting of: CR²⁰ and N;
D is N;
X, Y and Z are CR²⁰;
where each R²⁰ is independently selected from the group consisting of: hydrogen, alkyl, substituted alkyl, cycloalkyl, substituted cycloalkyl, —C(O)OR¹⁰,
where, R¹⁰ is selected from a group consisting of: hydrogen, C₁-C₄alkyl, and C₁-C₁₂aryl, and R¹¹ and R¹² are independently selected from a group consisting of: hydrogen, C₁-C₄alkyl, and C₁-C₁₂aryl;

provided that one and only one of A and B is N,

and/or a pharmaceutically acceptable salt, hydrate, solvate or pro-drug thereof.

Included among the novel compounds useful in the present invention are:
(5Z)-2-[(2,6-dichlorophenyl)amino]-5-(pyrazolo[1,5-a]pyridin-5-ylmethylidene)-1,3-thiazol-4(5H)-one;
(5Z)-2-[(2,6-dichlorophenyl)amino]-5-(imidazo[1,2-a]pyridin-6-ylmethylidene)-1,3-thiazol-4(5H)-one;
methyl 5-{(Z)-[2-[(2,6-dichlorophenyl)amino]-4-oxo-1,3-thiazol-5(4H)-ylidene]methyl}pyrazolo[1,5-a]pyridine-3-carboxylate;
ethyl 6-{(Z)-[2-[(2,6-dichlorophenyl)amino]-4-oxo-1,3-thiazol-5(4H)-ylidene]methyl}imidazo[1,2-a]pyridine-3-carboxylate;
N-(4-chloro-3-{[(5Z)-5-(imidazo[1,2-a]pyridin-6-ylmethylidene)-4-oxo-4,5-dihydro-1,3-thiazol-2-yl]amino}phenyl)cyclobutanecarboxamide
N-(4-chloro-3-{[(5Z)-4-oxo-5-(pyrazolo[1,5-a]pyridin-5-ylmethylidene)-4,5-dihydro-1,3-thiazol-2-yl]amino}phenyl)cyclobutanecarboxamide;
N-(4-chloro-3-{[(5Z)-4-oxo-5-(pyrazolo[1,5-a]pyridin-5-ylmethylidene)-4,5-dihydro-1,3-thiazol-2-yl]amino}phenyl)-2-methylpropanamide;
N-(4-chloro-3-{[(5Z)-5-(imidazo[1,2-a]pyridin-6-ylmethylidene)-4-oxo-4,5-dihydro-1,3-thiazol-2-yl]amino}phenyl)-2-methylpropanamide; and
N-(4-chloro-3-{[(5Z)-4-oxo-5-(pyrazolo[1,5-a]pyridin-5-ylmethylidene)-4,5-dihydro-1,3-thiazol-2-yl]amino}phenyl)-2,2-dimethylpropanamide;
   and/or a pharmaceutically acceptable salt, solvate, hydrate or pro-drug thereof.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Compounds of Formula I are included in the pharmaceutical compositions of the invention and used in the methods of the invention.

By the term “aryl” as used herein, unless otherwise defined, is meant a cyclic or polycyclic aromatic ring containing from 1 to 14 carbon atoms and optionally containing from one to five heteroatoms, provided that when the number of carbon atoms is 1 the aromatic ring contains at least four heteroatoms, when the number of carbon atoms is 2 the aromatic ring contains at least three heteroatoms, when the number of carbons is 3 the aromatic ring contains at least two heteroatoms and when the number of carbon atoms is 4 the aromatic ring contains at least one heteroatom.

By the term “C₁-C₁₂aryl” as used herein, unless otherwise defined, is meant phenyl, naphthalene, 3,4-methylenedioxyphenyl, pyridine, biphenyl, quinoline, pyrimidine, quinazoline, thiophene, thiazole, furan, pyrrole, pyrazole, imidazole, indole, indene, pyrazine, 1,3-dihydro-2H-benzimidazol, benzimidazol, benzothiophene, tetrahydrobenzothiophene and tetrazole.

The term “substituted” as used herein, unless otherwise defined, is meant that the subject chemical moiety has one or more substituents, suitably 1 to 5, selected from the group consisting of: alkyl, cycloalkyl, cycloalkyl containing from 1 to 4 heteroatoms, acyloxy, aryloxy, hydroxy, alkoxy, oxo, cyano, amino, alkylamino, dialkylamino, trifluoromethyl, —SO₂NR⁶¹R⁶², —N-acylamino, —CO₂R⁶⁰, —NC(O)R⁷⁰, halogen, aryl, aryl substituted with one to five substituents selected from a group consisting of: alkyl, C₁-C₆cycloalkyl, hydroxy, alkoxy, oxo, cyano, amino, alkylamino, dialkylamino, trifluoromethyl, —SO₂NR⁶¹R⁶², N-acylamino, —CO₂R⁶⁰, —NC(O)R⁷⁰, and halogen, where R⁶¹, R⁶², R⁶⁰ and R⁷⁰ are each independently selected from a group consisting of: hydrogen, cycloalkyl, aryl, substituted aryl, heteroaryl, substituted heteroaryl and C₁-C₄alkyl.

By the term “alkoxy” as used herein is meant —Oalkyl where alkyl is as described herein including —OCH₃ and —OC(CH₃)₂CH₃.

The term “cycloalkyl” as used herein unless otherwise defined, is meant a nonaromatic, unsaturated or saturated, cyclic or polycyclic C₃-C₁₂.

Examples of cycloalkyl and substituted cycloalkyl substituents as used herein include: cyclohexyl, aminocyclohexyl, cyclobutyl, aminocyclobutyl, 4-hydroxy-cyclohexyl, 2-ethylcyclohexyl, propyl-4-methoxycyclohexyl, 4-methoxycyclohexyl, 4-carboxycyclohexyl, cyclopropyl, aminocyclopentyl, and cyclopentyl.

The term “cycloalkyl containing from 1 to 4 heteroatoms” and the term “cycloalkyl containing from 1 to 3 heteroatoms” as used herein unless otherwise defined, is meant a nonaromatic, unsaturated or saturated, cyclic or polycyclic ring containing from 1 to 12 carbons and containing from one to four heteroatoms or from one to three heteroatoms (respectively), provided that when the number of carbon atoms is 1 the aromatic ring contains at least four heteroatoms (applicable only where “cycloalkyl containing from 1 to 4 heteroatoms” is indicated), when the number of carbon atoms is 2 the aromatic ring contains at least three heteroatoms, when the number of carbon atoms is 3 the nonaromatic ring contains at least two heteroatoms and when the number of carbon atoms is 4 the nonaromatic ring contains at least one heteroatom.

Examples of cycloalkyl containing from 1 to 4 heteroatoms, cycloalkyl containing from 1 to 3 heteroatoms, substituted cycloalkyl containing from 1 to 4 heteroatoms and substituted cycloalkyl containing from 1 to 3 heteroatoms as used herein include: piperidine, piperazine, pyrrolidine, 3-methylaminopyrrolidine, piperazine, tetrazole, hexahydrodiazepine and morpholine.

By the term “acyloxy” as used herein is meant —OC(O)alkyl where alkyl is as described herein. Examples of acyloxy substituents as used herein include: —OC(O)CH₃, —OC(O)CH(CH₃)₂ and —OC(O)(CH₂)₃CH₃.

By the term “N-acylamino” as used herein is meant —N(H)C(O)alkyl, where alkyl is as described herein. Examples of N-acylamino substituents as used herein include: —N(H)C(O)CH₃, —N(H)C(O)CH(CH₃)₂ and —N(H)C(O)(CH₂)₃CH₃.

By the term “aryloxy” as used herein is meant —Oaryl where aryl is phenyl, naphthyl, 3,4-methylenedioxyphenyl, pyridyl or biphenyl optionally substituted with one or more substituents selected from the group consisting of: alkyl, hydroxyalkyl, alkoxy, trifluoromethyl, acyloxy, amino, N-acylamino, hydroxy, —(CH₂)_{g}C(O)OR⁶⁵, S(O)ₙR⁶⁵, nitro, cyano, halogen and protected —OH, where g is 0-6, R⁶⁵ is selected from a group consisting of hydrogen and alkyl, and n is 0-2. Examples of aryloxy substituents as used herein include: phenoxy, 4-fluorophenyloxy and biphenyloxy.

By the term “heteroatom” as used herein is meant oxygen, nitrogen or sulfur.

By the term “halogen” as used herein is meant a substituent selected from a group consisting of: bromide, iodide, chloride and fluoride.

By the term “alkyl” and derivatives thereof and in all carbon chains as used herein, including alkyl chains defined by the term “—(CH₂)ₙ”, “—(CH₂)ₘ” and the like, is meant a linear or branched, saturated or unsaturated hydrocarbon chain, and unless otherwise defined, the carbon chain will contain from 1 to 12 carbon atoms.

Examples of alkyl and substituted alkyl substituents as used herein include:

—CH₃, —CH₂—CH₃, —CH₂—CH₂—CH₃, —CH(CH₃)₂, —CH₂—CH₂—C(CH₃)₃, —CH₂—CF₃, —C≡C—C(CH₃)₃, —C≡C—CH₂—OH, cyclopropylmethyl, —CH₂—C(CH₃)₂—CH₂—NH₂, —C≡C—C₆H₅, —C≡C—C(CH₃)₂—OH, —CH₂—CH(OH)—CH(OH)—CH(OH)—CH(OH)—CH₂—OH, piperidinylmethyl, methoxyphenylethyl, —C(CH₃)₃, —(CH₂)₃—CH₃, —CH₂—CH(CH₃)₂, —CH(CH₃)—CH₂—CH₃, —CH═CH₂, and —C≡C—CH₃.

By the term “treating” and derivatives thereof as used herein, is meant prophylatic and therapeutic therapy.

As used herein, the term “optionally” means that the subsequently described event(s) may or may not occur, and includes both event(s), which occur, and events that do not occur.

As used herein, the crisscrossed double bond indicated by the symbol [symbol] denotes Z and/or E stereochemistry around the double bond. In other words a compound of Formula I can be either in the Z or E stereochemistry around this double bond, or a compound of Formula I can also be in a mixture of Z and E stereochemistry around the double bond. However, in Formula I, the preferred compounds have Z stereochemistry around the double bond to which radical Q is attached.

A person of ordinary skill can readily appreciate that certain substituents may cause the double bonds of Q in Formula I to shift within the bicyclic ring. An example of such substituent is: oxo.

The compounds of Formula I naturally may exist in one tautomeric form or in a mixture of tautomeric forms. For example, for sake simplicity, compounds of Formula I are expressed in one tautomeric form, usually as an exo form, i.e.

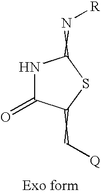

However, a person of ordinary skill can readily appreciate, the compounds of formula can also exist in endo forms.

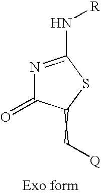

The present invention contemplates all possible tautomeric forms.

Certain compounds described herein may contain one or more chiral atoms, or may otherwise be capable of existing as two enantiomers, or two or more diastereoisomers. Accordingly, the compounds of this invention include mixtures of enantiomers/diastereoisomers as well as purified enantiomers/diastereoisomers or enantiomerically/diastereoisomerically enriched mixtures. Also included within the scope of the invention are the individual isomers of the compounds represented by Formula I above as well as any wholly or partially equilibrated mixtures thereof. The present invention also covers the individual isomers of the compounds represented by the formula above as mixtures with isomers thereof in which one or more chiral centers are inverted. Further, an example of a possible tautomer is an oxo substituent in place of a hydroxy substituent. Also, as stated above, it is understood that all tautomers and mixtures of tautomers are included within the scope of the compounds of Formula I.

Compounds of Formula I are included in the pharmaceutical compositions of the invention and used in the methods of the invention. Where a —COOH or —OH group is present, pharmaceutically acceptable esters can be employed, for example methyl, ethyl, pivaloyloxymethyl, and the like for —COOH, and acetate maleate and the like for —OH, and those esters known in the art for modifying solubility or hydrolysis characteristics, for use as sustained release or pro-drug formulations.

The novel compounds of Formula I are prepared as shown in Schemes I and II below, or by analogous methods, wherein the ‘Q’ and ‘R’ substituents are as defined in Formula I respectively and provided that the ‘Q’ and ‘R’ substituents do not include any such substituents that render inoperative the processes of Schemes I to II. All of the starting materials are commercially available or are readily made from commercially available starting materials by those of skill in the art.

### General Schemes

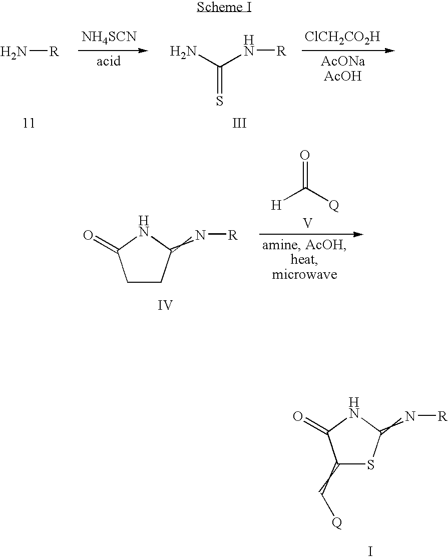

Briefly in Scheme 1, a mixture of aniline derivative of formula II (1 equivalent) and NH4SCN (about 1.3 equivalent) in an acid (typically 4N—HCl) is heated to reflux at about 110° C. for 6 hours. After cooling, the mixture is treated with H₂O, which process usually forms a solid, followed by desiccation in vacuo to give a compound of formula III.

A mixture of formula III compound, ClCH₂CO₂H (1 equivalent), and AcONa (1 equivalent) in ACOH is heated to reflux at around 110° C. for about 4 h. The mixture is poured onto water thereby a solid is typically formed, which is isolated by filtration. The solid is washed with a solvent such as MeOH to afford a compound of formula IV.

A mixture of formula IV compound, an aldehyde of formula V (1 equivalent), an amine such as piperidine, or piperidine acetate, and optionally acetic acid in ethanol is heated in a microwave reactor at about 150° C. for about 1 hour. After cooling, a small portion of water is added until the solid forms. The solid is filtered and washed with a solvent such as MeOH, followed by desiccation in vacuo to afford a target product of Formula I.

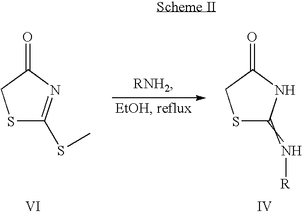

Scheme II shows an alternative synthesis of the intermediate IV. Briefly in Scheme 2, a mixture of the known thiazolinone VI and aniline derivative RNH₂ in ethanol is heated under reflux to give the intermediate IV after appropriate work-up.

International application NO. PCT/US2003/037658, having an international filing date of Nov. 18, 2003; which also has International Publication Number WO2004/047760 and an International Publication date of Jun. 10, 2004, describes general procedures of making intermediates IV.

Intermediates IV used in the following examples can be prepared according to Scheme I and II, or the methods described in PCT/US2003/037658.

In Schemes I and II, the meaning of R and Q are as defined in Formula I.

In other embodiments, additional compounds of the invention can also be synthesized whereby a compound of Formula I is first made by a process of Scheme 1 or 2 (or a variant thereof), and Q and R radicals in compounds of Formula I thus made are further converted by routine organic reaction techniques into different Q and R groups.

By the term “co-administering” and derivatives thereof as used herein is meant either simultaneous administration or any manner of separate sequential administration of a YAK3 inhibiting compound, as described herein, and a further active ingredient or ingredients, known to be useful in treating diseases of the hematopoietic system, particularly anemias, including EPO or a derivative thereof. The term further active ingredient or ingredients, as used herein, includes any compound or therapeutic agent known to or that demonstrates advantageous properties when administered to a patient in need of treatment for diseases of the hematopoietic system, particularly anemias. Preferably, if the administration is not simultaneous, the compounds are administered in a close time proximity to each other. Furthermore, it does not matter if the compounds are administered in the same dosage form, e.g. one compound may be administered topically and another compound may be administered orally.

Because the pharmaceutically active compounds of the present invention are active as YAK3 inhibitors they exhibit therapeutic utility in treating diseases of the hematopoietic system, particularly anemias.

The pharmaceutically active compounds within the scope of this invention are useful as YAK inhibitors in mammals, particularly humans, in need thereof.

The present invention therefore provides a method of treating diseases of the hematopoietic system, particularly anemias and other conditions requiring YAK inhibition, which comprises administering an effective compound of Formula I or a pharmaceutically acceptable salt, hydrate, solvate or pro-drug thereof. The compounds of Formula I also provide for a method of treating the above indicated disease states because of their ability to act as YAK inhibitors. The drug may be administered to a patient in need thereof by any conventional route of administration, including, but not limited to, intravenous, intramuscular, oral, subcutaneous, intradermal, and parenteral.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-100 mg/kg of active compound, preferably 0.001-50 mg/kg. When treating a human patient in need of a YAK inhibitor, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages is preferred. Parenteral administration, at high dosages, however, also can be used when safe and convenient for the patient.

Optimal dosages to be administered may be readily determined by those skilled in the art, and will vary with the particular YAK inhibitor in use, the strength of the preparation, the mode of administration, and the advancement of the disease condition. Additional factors depending on the particular patient being treated will result in a need to adjust dosages, including patient age, weight, diet, and time of administration.

The method of this invention of inducing YAK inhibitory activity in mammals, including humans, comprises administering to a subject in need of such activity an effective YAK inhibiting amount of a pharmaceutically active compound of the present invention.

The invention also provides for the use of a compound of Formula I in the manufacture of a medicament for use as a YAK inhibitor.

The invention also provides for the use of a compound of Formula I in the manufacture of a medicament for use in therapy.

The invention also provides for the use of a compound of Formula I in the manufacture of a medicament for use in treating diseases of the hematopoietic system, particularly anemias.

The invention also provides for a pharmaceutical composition for use as a YAK inhibitor which comprises a compound of Formula I and a pharmaceutically acceptable carrier.

The invention also provides for a pharmaceutical composition for use in the treatment of diseases of the hematopoietic system, particularly anemias which comprises a compound of Formula I and a pharmaceutically acceptable carrier.

No unacceptable toxicological effects are expected when compounds of the invention are administered in accordance with the present invention.

In addition, the pharmaceutically active compounds of the present invention can be co-administered with further active ingredients, such as other compounds known to treat diseases of the hematopoietic system, particularly anemias, or compounds known to have utility when used in combination with a YAK inhibitor.

Without further elaboration, it is believed that one skilled in the art can, using the preceding description, utilize the present invention to its fullest extent. The following Examples are, therefore, to be construed as merely illustrative and not a limitation of the scope of the present invention in any way.

For ease of illustration, the regiochemistry around the double bonds in the chemical formula in the Examples are drawn as fixed for ease of representation; however, a skilled in the art will readily appreciate that the compounds will naturally assume more thermodynamically stable structure around the C═N (the imine) double bond if it exits as exo form. Further compounds can also exit in endo form. As stated before, the invention contemplates both endo and exo forms as well as both regioisomers around the exo imine bond. Further it is intended that both E and Z isomers are encompassed around the C═C double bond.

### EXAMPLE 1

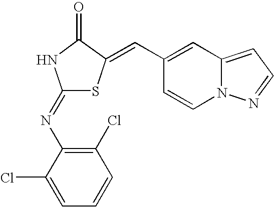

### (5Z)-2-[(2,6-dichlorophenyl)amino]-5-(pyrazolo[1,5-a]pyridin-5-ylmethylidene)-1,3-thiazol-4(5H)-one

A solution of pyrazolo[1,5-a]pyridine-5-carbaldehyde (prepared by the method of Bettinetti, L.; Schlotter, K.; Hübner, H.; Gmeiner, P. J. Med. Chem., 2002, 45, 21, 4594-4597)(0.040 g, 0.267 mmol) and (2Z)-2-[(2,6-dichlorophenyl)imino]-1,3-thiazolidin-4-one (0.070 g, 0.267 mmol) in ethanol (2.0 mL) was treated with piperidine (0.020 mL, 0.267 mmol) and then heated to 150° C. for 1 h in a Biotage Initiator microwave synthesizer. The reaction mixture was allowed to cool to ambient temperature and acidified with 1N aqueous hydrochloric acid. The resulting precipitate was filtered, washed with water and dried in vacuo to afford the title compound as a bright yellow powder (0.052 g; 50%). ¹H NMR (400 MHz, DMSO-d₆) δ ppm 13.0 (s, 1H), 8.70 (d, J=7.3 Hz, 1H), 8.07 (d, J=2.3 Hz, 1H), 7.93 (s, 1H), 7.76 (s, 1H), 7.58 (d, J=8.1 Hz, 2H), 7.24 (t, J=8.2 Hz, 1H), 6.94 (dd, J=7.3, 1.8 Hz, 1H), 6.82 (d, J=1.8 Hz, 1H). MS (ES+) m/e 389 [M+H]⁺.

### EXAMPLE 2

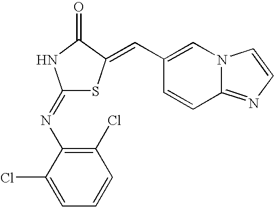

### (5Z)-2-[(2,6-dichlorophenyl)amino]-5-(imidazo[1,2-a]pyridin-6-ylmethylidene)-1,3-thiazol-4(5H)-one

Following the procedure of Example 1, except substituting imidazo[1,2a]pyridine-6-carbaldehyde (prepared by the method of Yamanaka, M.; Miyake, K.; Suda, S.; Ohhara, H.; Ogawa, T. Chem. Pharm. Bull. 1991, 39, 6, 1556-1567) for pyrazolo[1,5-a]pyridine-5-carbaldehyde, the title compound was obtained as a yellow powder. ¹H NMR (400 MHz, METHANOL-d₄) δ ppm 8.74 (s, 1H), 7.94 (s, 1H), 7.72 (s, 1H), 7.63 (s, 1H), 7.60 (d, J=9.3 Hz, 1H), 7.47 (d, J=8.1 Hz, 2H), 7.42 (dd, J=9.5, 1.4 Hz, 1H), 7.18 (t, J=8.2 Hz, 1H). MS (ES+) m/e 389 [M+H]⁺.

### EXAMPLE 3

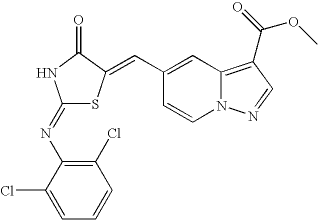

### Methyl 5-{(Z)-[2-[(2,6-dichlorophenyl)amino]-4-oxo-1,3-thiazol-5(4H)-ylidene]methyl}pyrazolo[1,5-a]pyridine-3-carboxylate

### a) Methyl 5-formylpyrazolo[1,5-a]pyridine-3-carboxylate

A solution of methyl 5-(hydroxymethyl)pyrazolo[1,5-a]pyridine-3-carboxylate (prepared by the method of Bettinetti, L.; Schlotter, K.; Hübner, H.; Gmeiner, P. J. Med. Chem., 2002, 45, 21, 4594-4597)(0.110 g, 0.538 mmol) in methylene chloride (2.0 mL) was treated with manganese dioxide (0.460 g, 5.38 mmol) at ambient temperature. Following stirring for 12 h, the solution was filtered, concentrated in vacuo, and purified via flash column chromatography (10% methanol in dichloromethane) to afford the title compound as a white solid (0.111 g, 100%). ¹H NMR (400 MHz, DMSO-d₆) δ ppm 10.1 (s, 1H), 8.70 (s, 1H), 8.62 (d, J=7.1 Hz, 1H), 8.52 (s, 1H), 7.47 (dd, J=7.1, 1.8 Hz, 1H), 4.00 (s, 3H). MS (ES+) m/e 205 [M+H]⁺.

### b) Methyl 5-{(Z)-[2-[(2,6-dichlorophenyl)amino]-4-oxo-1,3-thiazol-5(4H)-ylidene]methyl}pyrazolo[1,5-a]pyridine-3-carboxylate

Following the procedure of Example 1, except substituting the compound from example 3a) for pyrazolo[1,5-a]pyridine-5-carbaldehyde, the title compound was obtained as a yellow powder. ¹H NMR (400 MHz, DMSO-d₆) δ ppm 8.74 (d, J=7.1 Hz, 1H), 8.42 (s, 1H), 8.16 (br. s., 1H), 7.76 (s, 1H), 7.49 (d, J=8.1 Hz, 2H), 7.11-7.23 (m, 2H), 3.73 (s, 3H). MS (ES+) m/e 447 [M+H]⁺.

### EXAMPLE 4

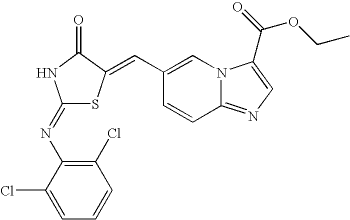

### Ethyl

### 6-{(Z)-[2-[(2,6-dichlorophenyl)amino]-4-oxo-1,3-thiazol-5(4H)-ylidene]methyl}imidazo[1,2-a]pyridine-3-carboxylate

### a) 3-[(ethyloxy)carbonyl]imidazo[1,2-a]pyridine-6-carboxylic acid

A solution of concentrated aqueous sulfuric acid (0.05 mL) in ethanol (2.5 mL) at 0° C. was treated with ethyl 2-chloro-3-oxopropanoate potassium salt (prepared by the method of Ikemoto, T.; Kawamoto, T.; Tomimatsu, K.; Takatani, M; Wakimasu, M. Tetrahedron 2000, 56, 40, 7915) (0.466 g, 3.10 mmol) and 6-amino-3-pyridinecarboxylic acid (0.138 g, 1.00 mmol) and then heated to reflux for 18 h. Upon cooling, the solution was concentrated in vacuo, the residue was poured into 5 mL of water and extracted thrice with 10 mL portions of ethyl acetate. The aqueous layer was neutralized with 1N aqueous NaOH and further extracted with ethyl acetate. The organic extracts were washed with water, dried over MgSO₄, filtrated and concentrated in vacuo to afford the title compound (0.234 g; 100%). MS (ES+) m/e 235 [M+H]⁺.

### b) Ethyl 6-(hydroxymethyl)imidazo[1,2-a]pyridine-3-carboxylate

A solution of the compound from Example 4a) (0.234 g, 1.0 mmol) in tetrahydrofuran (1.0 mL) was cooled to −18° C. in an ice-salt bath. A solution of borane-tetrahydrofuran (1.0 mL, 1.0 mmol, 1M in THF) was added dropwise over 5 minutes. The resulting clear solution was stirred well and the ice-salt bath was allowed to warm slowly to ambient temperature over 8 h. The reaction was quenched with water at 0° C., followed by extraction with ethyl ether (4×20 mL), concentration in vacuo, and purification via flash column chromatography (10% methanol in chloroform) to afford the title compound as a yellow oil (0.102 g; 46%). ¹H NMR (400 MHz, METHANOL-d₄) δ ppm 9.31 (br. s., 1H), 8.22 (s, 1H), 7.69 (d, J=9.1 Hz, 1H), 7.57 (dd, J=9.2, 1.6 Hz, 1H), 4.72 (s, 2H), 4.43 (q, J=7.1 Hz, 2H), 1.43 (t, J=7.1 Hz, 3H). MS (ES+) m/e 221 [M+H]⁺.

### c) Ethyl 6-formylimidazo[1,2-a]pyridine-3-carboxylate

A solution of the compound from Example 4b) (0.102 g, 0.463 mmol) in dichloromethane (5.0 mL) was treated with manganese dioxide (0.403 g, 4.63 mmol). Following stirring for 8 h at ambient temperature, the mixture was filtered through a pad of celite and washed three times with dichloromethane. The solution was concentrated in vacuo to afford the title compound as a yellow solid (0.055 g; 54%). ¹H NMR (400 MHz, CHLOROFORM-d) δ ppm 10.1 (s, 1H), 9.86 (d, J=1.8 Hz, 1H), 8.39 (s, 1H), 7.88-7.96 (m, 1H), 7.77-7.85 (m, 1H), 4.47 (q, J=7.1 Hz, 2H), 1.46 (t, J=7.1 Hz, 3H). MS (ES+) m/e 219 [M+H]⁺.

### d) Ethyl

### 6-{(Z)-[2-[(2,6-dichlorophenyl)amino]-4-oxo-1,3-thiazol-5(4H)-ylidene]methyl}imidazo[1,2-a]pyridine-3-carboxylate

Following the procedure of Example 1, except substituting the compound from example 4c) for pyrazolo[1,5-a]pyridine-5-carbaldehyde, the title compound was obtained as a white powder. ¹H NMR (400 MHz, DMSO-d₆) δ ppm 13.1 (br. s., 1H), 9.39 (s, 1H), 8.29-8.43 (m, 1H), 7.80-7.92 (m, 2H), 7.65 (d, J=9.3 Hz, 1H), 7.58 (d, J=8.1 Hz, 2H), 7.24 (t, J=8.1 Hz, 1H), 4.30 (q, J=6.8 Hz, 2H), 1.27 (t, J=7.1 Hz, 3H). MS (ES+) m/e 461 [M+H]⁺.

### EXAMPLE 6

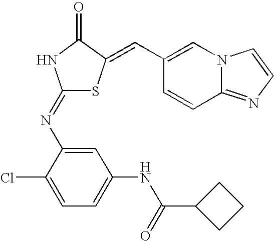

### N-(4-chloro-3-{[(5Z)-5-(imidazo[12-a]pyridin-6-ylmethylidene)-4-oxo-4,5-dihydro-1,3-thiazol-2-yl]amino}phenyl)cyclobutanecarboxamide

Following the procedure of Example 1, except substituting imidazo[1,2a]pyridine-6-carbaldehyde (prepared by the method of Yamanaka, M.; Miyake, K.; Suda, S.; Ohhara, H.; Ogawa, T. Chem. Pharm. Bull. 1991, 39, 6, 1556-1567) for pyrazolo[1,5-a]pyridine-5-carbaldehyde and N-(4-chloro-3-{[(2Z)-4-oxo-1,3-thiazolidin-2-ylidene]amino}phenyl)cyclobutanecarboxamide for (2Z)-2-[(2,6-dichlorophenyl)imino]-1,3-thiazolidin-4-one, the title compound was obtained as a red solid. ¹H NMR (400 MHz, METHANOL-d₄) δ ppm 9.01 (s, 1H), 8.29 (d, J=1.8 Hz, 1H), 8.06 (dd, J=5.3, 1.8 Hz, 2H), 7.95 (d, J=9.3 Hz, 1H), 7.74 (s, 1H), 7.52 (d, J=2.0 Hz, 1H), 7.38 (d, J=8.8 Hz, 1H), 7.29 (dd, J=14.9, 2.3 Hz, 1H), 3.25-3.37 (m, 1H), 2.26-2.40 (m, 2H), 2.12-2.26 (m, 2H), 1.96-2.11 (m, 1H), 1.88 (q, J=9.3 Hz, 1H). MS (ES+) m/e 452 [M+H]⁺.

### EXAMPLE 6

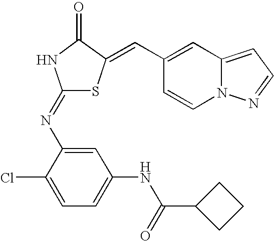

### N-(4-chloro-3-{[(5Z)-4-oxo-5-(pyrazolo[1,5-a]pyridin-5-ylmethylidene)-4,5-dihydro-1,3-thiazol-2-yl]amino}phenyl)cyclobutanecarboxamide

A solution of pyrazolo[1,5-a]pyridine-5-carbaldehyde (prepared by the method of Bettinetti, L.; Schlotter, K.; Hübner, H.; Gmeiner, P. J. Med. Chem., 2002, 45, 21, 4594-4597)(0.040 g, 0.267 mmol) and N-(4-chloro-3-{[(2Z)-4-oxo-1,3-thiazolidin-2-ylidene]amino}phenyl)cyclobutanecarboxamide (0.173 g, 0.536 mmol) in ethanol (2.0 mL) was treated with piperidinium acetate (0.536 mL, 0.536 mmol, 1M solution in ethanol) and then heated to 150° C. for 1 h in a Biotage Initiator microwave synthesizer. The reaction mixture was allowed to cool to ambient temperature and treated with water. The resulting precipitate was filtered, washed with water, hexanes, and methanol to afford the title compound as a yellow solid (0.075 g; 62%). ¹H NMR (400 MHz, DMSO-d₆) δ ppm 12.8 (br. s., 1H), 9.93 (s, 1H), 8.71 (d, J=7.1 Hz, 1H), 8.06 (d, J=2.3 Hz, 1H), 7.93 (s, 1H), 7.71 (s, 1H), 7.31-7.53 (m, 3H), 6.97 (dd, J=7.3, 1.8 Hz, 1H), 6.81 (d, J=1.5 Hz, 1H), 2.16-2.29 (m, 2H), 2.02-2.15 (m, 2H), 1.85-2.00 (m, 1H), 1.72-1.86 (m, 1H), 0.90-1.16 (m, 1H). MS (ES+) m/e 452 [M+H]⁺.

### EXAMPLE 7

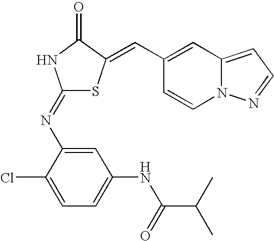

### N-(4-chloro-3-{[(5Z)-4-oxo-5-(pyrazolo[1,5-a]pyridin-5-ylmethylidene)-4,5-dihydro-1,3-thiazol-2-yl]amino}phenyl)-2-methylpropanamide

Following the procedure of Example 6, except substituting N-(4-chloro-3-{[(2Z)-4-oxo-1,3-thiazolidin-2-ylidene]amino}phenyl)-2-methylpropanamide for N-(4-chloro-3-{[(2Z)-4-oxo-1,3-thiazolidin-2-ylidene]amino}phenyl)cyclobutanecarboxamide, the title compound was obtained as a bright yellow solid. ¹H NMR (400 MHz, DMSO-d₆) δ ppm 12.8 (br. s., 1H), 10.0 (s, 1H), 8.70 (d, J=7.6 Hz, 1H), 8.05 (d, J=2.3 Hz, 1H), 7.92 (s, 1H), 7.70 (s, 1H), 7.44 (d, J=2.5 Hz, 3H), 6.96 (dd, J=7.5, 1.9 Hz, 1H), 6.80 (d, J=1.5 Hz, 1H), 2.53-2.62 (m, 1H), 1.10 (s, 3H), 1.08 (s, 3H). MS (ES+) m/e 440 [M+H]⁺.

### EXAMPLE 8

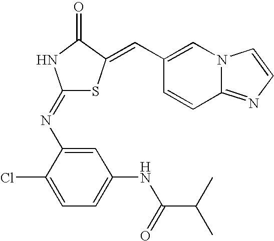

### N-(4-chloro-3-[{(5Z)-5-(imidazo[1,2-a]pyridin-6-ylmethylidene)-4-oxo-4,5-dihydro-1,3-thiazol-2-yl]amino}phenyl)-2-methylpropanamide

Following the procedure of Example 6, except substituting imidazo[1,2-a]pyridine-6-carbaldehyde (prepared by the method of Yamanaka, M.; Miyake, K.; Suda, S.; Ohhara, H.; Ogawa, T. Chem Pharm. Bull. 1991, 39, 6, 1556-1567) for pyrazolo[1,5-a]pyridine-5-carbaldehyde and N-(4-chloro-3-{[(2Z)-4-oxo-1,3-thiazolidin-2-ylidene]amino}phenyl)-2-methylpropanamide for N-(4-chloro-3-{[(2Z)-4-oxo-1,3-thiazolidin-2-ylidene]amino}phenyl)cyclobutanecarboxamide, the title compound was obtained as a light orange solid. ¹H NMR (400 MHz, DMSO-d₆) δ ppm 12.7 (br. s., 1H), 10.0 (s, 1H), 8.91 (s, 1H), 8.06 (s, 1H), 7.70 (s, 1H), 7.58-7.67 (m, 2H), 7.45 (d, J=2.3 Hz, 3H), 7.33 (dd, J=9.6, 1.8 Hz, 1H), 2.52-2.66 (m, 1H), 1.10 (s, 3H), 1.09 (s, 3H). MS (ES+) m/e 440 [M+H]⁺.

### EXAMPLE 9

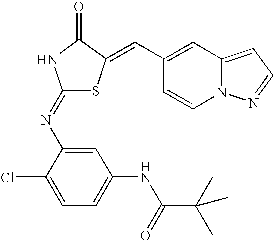

### N-(4-chloro-3-{[(5Z)-4-oxo-5-(pyrazolo[1,5-a]pyridin-5-ylmethylidene)-4,5-dihydro-1,3-thiazol-2-yl]amino}phenyl)-2,2-dimethylpropanamide

Following the procedure of Example 6, except substituting N-(4-chloro-3-{[(2Z)-4-oxo-1,3-thiazolidin-2-ylidene]amino}phenyl)-2,2-dimethylpropanamide for N-(4-chloro-3-{[(2Z)-4-oxo-1,3-thiazolidin-2-ylidene]amino}phenyl)cyclobutanecarboxamide, the title compound was obtained as a bright yellow solid. ¹H NMR (400 MHz, DMSO-d₆) δ ppm 12.74 (br. s., 1H), 9.36 (s, 1H), 8.70 (d, J=7.3 Hz, 1H), 8.05 (d, J=2.0 Hz, 1H), 7.92 (s, 1H), 7.70 (s, 1H), 7.48-7.55 (m, 2H), 7.40-7.47 (m, 1H), 6.96 (dd, J=7.3, 2.0 Hz, 1H), 6.80 (d, J=1.8 Hz, 1H), 1.21 (s, 9H). MS (ES+) m/e 454 [M+H]⁺.

### EXAMPLE 10

### Capsule Composition

An oral dosage form for administering the present invention is produced by filing a standard two piece hard gelatin capsule with the ingredients in the proportions shown in Table I, below.

TABLE I

| INGREDIENTS | AMOUNTS | |
|---|---|---|
| Compound of example 1 | 25 | mg |
| Lactose | 55 | mg |
| Talc | 16 | mg |
| Magnesium Stearate | 4 | mg |

### EXAMPLE 11

### Injectable Parenteral Composition

An injectable form for administering the present invention is produced by stirring 1.5% by weight of compound of example 1 in 10% by volume propylene glycol in water.

### EXAMPLE 12

### Tablet Composition

The sucrose, calcium sulfate dihydrate and an YAK inhibitor as shown in Table II below, are mixed and granulated in the proportions shown with a 10% gelatin solution. The wet granules are screened, dried, mixed with the starch, talc and stearic acid, screened and compressed into a tablet.

TABLE II

| INGREDIENTS | AMOUNTS | |
|---|---|---|
| Compound of example 1 | 20 | mg |
| calcium sulfate dihydrate | 30 | mg |
| Sucrose | 4 | mg |
| Starch | 2 | mg |
| Talc | 1 | mg |
| stearic acid | 0.5 | mg |

### Biological Methods and Data

Because the compounds of the present invention are active as inhibitors of YAK3 they exhibit therapeutic utility in treating diseases associated with YAK3 activity, including but not limited to, anemia, anemias due to renal insufficiency or to chronic disease, such as autoimmunity, HIV, or cancer, and drug-induced anemias, myelodysplastic syndrome, aplastic anemia and myelosuppression, and cytopenia.

Substrate phosphorylation assays are carried out as follows:

YAK3 Scintillation Proximity Assays Using Ser164 of Myelin Basic Protein as the phosphoacceptor

The source of Ser164 substrate peptide The biotinylated Ser164, S164A peptide(Biotinyl-LGGRDSRAGS*PMARR—OH), sequence derived from the C-terminus of bovine myelin basic protein (MBP) with Ser162 substituted as Ala162, is purchased from California Peptide Research Inc. (Napa, Calif.), and its purity is determined by HPLC. Phosphorylation occurs at position 164 (marked S* above). The calculated molecular mass of the peptide is 2166 dalton. Solid sample is dissolved at 10 mM in DMSO, aliquoted, and stored at −20° C. until use.

The source of enzyme:

YAK3: Glutathione-S-Transferase (GST)-YAK3-His6 containing amino acid residues 124-526 of human YAK3 (aa 124-526 of SEQ ID NO 2. in U.S. Pat. No. 6,323,318) is purified from baculovirus expression system in Sf9 cells using Glutathione Sepharose 4B column chromatography followed by Ni-NTA-Agarose column chromatography. Purity greater than 65% typically is achieved. Samples, in 50 mM Tris, 150 mM NaCl, 10% glycerol, 0.1% Triton, 250 mM imidazole, 10 mM β-mercapto ethanol, pH 8.0. are stored at −80° C. until use.

Kinase assay of purified YAK3: Assays are performed in 96 well (Costar, Catalog No. 3789) or 384 well plates (Costar, Catalog No. 3705). Reaction (in 20, 25, or 40 μl volume) mix contained in final concentrations 25 mM Hepes buffer, pH 7.4; 10 mM MgCl2; 10 mM β-mercapto ethanol; 0.0025% Tween-20; 0.001 mM ATP, 0.1 δ Ci of [δ-33P]ATP; purified YAK3 (7-14 ng/assay; 4 nM final); and 4 μM Ser164 peptide. Compounds, titrated in DMSO, are evaluated at concentrations ranging from 50 μM to 0.5 nM. Final assay concentrations of DMSO do not exceed 5%, resulting in less than 15% loss of YAK3 activity relative to controls without DMSO. Reactions are incubated for 2 hours at room temperature and are stopped by a 75 ul addition of 0.19 μg Streptavidin Scintillation Proximity beads (Amersham Pharmacia Biotech, Catalog No. RPNQ 0007) in PBS, pH 7.4, 10 mM EDTA, 0.1% Triton X-100, 1 mM ATP. Under the assay conditions defined above, the Kₘ(apparent) for ATP is determined to be 7.2+−2.4 μM.

The data for compounds dose responses are plotted as % inhibition, calculated with the data reduction formula 100*(1-[U1-C2)/(C1-C2)]), versus concentration of compound, where U is the unknown value, C1 is the average control value obtained for DMSO, and C2 is the average control value obtained for 0.05M EDTA. DATA were fitted tot h curve described by: y=((Vmax*x)/(K+x)) were Vmax is the upper asymptote and K is the IC50. The results for each compound were recorded asplC50 calculated as follows: plC50=−Log 10(K).

Utility of the Present Invention

The compounds of Formula I are useful for treating or preventing disease states in which YAK3 proteins are implicated, especially diseases of the erythroid and hematopoietic systems, including but not limited to, anemias due to renal insufficiency or to chronic disease, such as autoimmunity, HIV, or cancer, and drug-induced anemias, myelodysplastic syndrome, aplastic anemia, myelosuppression, and cytopenia.

The compounds of Formula I are useful in treating diseases of the hematopoietic system, particularly anemias. Such anemias include an anemia selected from the group comprising: aplastic anemia and myelodysplastic syndrome. Such anemias also include those wherein the anemia is a consequence of a primary disease selected from the group consisting of: cancer, leukemia and lymphoma. Such anemias also include those wherein the anemia is a consequence of a primary disease selected from the group consisting of: renal disease, failure or damage. Such anemias include those wherein the anemia is a consequence of chemotherapy or radiation therapy, in particular wherein the chemotherapy is chemotherapy for cancer or AZT treatment for HIV infection. Such anemias include those wherein the anemia is a consequence of a bone marrow transplant or a stem cell transplant. Such anemias also include anemia of newborn infants. Such anemias also include those which are a consequence of viral, fungal, microbial or parasitic infection.

The compounds of Formula I are also useful for enhancing normal red blood cell numbers. Such enhancement is desirable for a variety of purposes, especially medical purposes such as preparation of a patient for transfusion and preparation of a patient for surgery.

While the preferred embodiments of the invention are illustrated by the above, it is to be understood that the invention is not limited to the precise instructions herein disclosed and that the right to all modifications coming within the scope of the following claims is reserved.

What is claimed is:

1. A compound of Formula I: 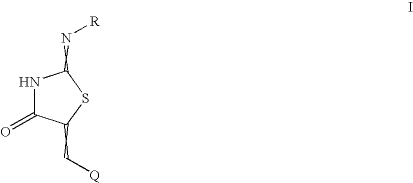
in which
R is selected from a group consisting of: aryl, substituted aryl, heteroaryl, substituted heteroaryl, alkyl, substituted alkyl, alkoxy, —N—R¹⁵, —O—R¹⁵, cycloalkyl, substituted cycloalkyl, cycloalkyl containing from 1 to 4 heteroatoms, substituted cycloalkyl containing from 1 to 4 heteroatoms,
where R¹⁵ is selected from a group consisting of: C₁-C₁₂aryl, substituted C₁-C₁₂aryl, alkyl, substituted alkyl, cycloalkyl, substituted cycloalkyl, cycloalkyl containing from 1 to 4 heteroatoms, substituted cycloalkyl containing from 1 to 4 heteroatoms; and
Q is 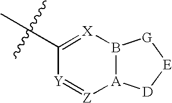
wherein,
A, B, D, E, and G together form a ring containing from 1 to 2 double bonds and from 1 to 4 nitrogens;
where,
A and B are independently selected from a group consisting of: C and N;
G, E, and D are independently selected from a group consisting of: CR²⁰, O, S, and N;
X, Y and Z are CR²⁰;
where each R²⁰ is independently selected from the group consisting of: hydrogen, halogen, amino, alkylamine, substituted alkylamine, dialkylamine, substituted dialkylamine, hydroxy, alkoxy, alkyl, substituted alkyl, aryl, substituted aryl, heteroaryl, substituted heteroaryl, arylamine, substituted arylamine, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected from a group consisting of: hydrogen, C₁-C₄alkyl, and aryl and R¹¹ and R¹² are independently selected from a group consisting of: hydrogen, C₁-C₄alkyl, and aryl;
provided that one and only one of A and B is N,
also provided that R is not unsubstituted phenyl;
and/or a pharmaceutically acceptable salt, hydrate, solvate or pro-drug thereof.

2. A compound according to claim 1 wherein R is substituted aryl; and/or a pharmaceutically acceptable salt, hydrate, solvate or pro-drug thereof.

3. A compound according to claim 1 wherein Q is: 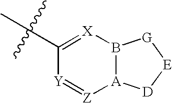
wherein,
R is selected from a group consisting of: aryl, substituted aryl, heteroaryl, and substituted heteroaryl;
A, B, D, E, and G together form a ring containing 2 double bonds and 2 nitrogens;
where,
A and B are independently selected from a group consisting of: C and N;
G, E, and D are independently selected from a group consisting of: CR²⁰ and N;
X, Y and Z are CR²⁰;
where each R²⁰ is independently selected from the group consisting of: hydrogen, halogen, amino, alkylamine, substituted alkylamine, dialkylamine, substituted dialkylamine, hydroxy, alkoxy, alkyl, substituted alkyl, aryl, substituted aryl, heteroaryl, substituted heteroaryl, arylamine, substituted arylamine, cycloalkyl, substituted cycloalkyl, cycloalkyl containing from 1 to 4 heteroatoms, substituted cycloalkyl containing from 1 to 4 heteroatoms, oxo, —C(O)OR¹⁰, —C(O)NR¹¹R¹², cyano, and nitrile,
where, R¹⁰ is selected from a group consisting of: hydrogen, C₁-C₄alkyl, and C₁-C₁₂aryl, and R¹¹ and R¹² are independently selected from a group consisting of:
hydrogen, C₁-C₄alkyl, and C₁-C₁₂aryl;
provided that one and only one of A and B is N,
also provided that R is not unsubstituted phenyl;
and/or a pharmaceutically acceptable salt, solvate, hydrate or pro-drug thereof.

4. A compound according to claim 3 wherein R is substituted aryl; and/or a pharmaceutically acceptable salt, hydrate, solvate or pro-drug thereof.

5. A compound according to claim 3 wherein D is N; R is C₁-C₁₂ substituted aryl; each R²⁰ is independently selected from the group consisting of: hydrogen, alkyl, substituted alkyl, cycloalkyl, substituted cycloalkyl, —C(O)OR¹⁰, where R¹⁰ is defined according to claim 3.

6. A compound according to claim 1 wherein D is N.

7. A compound according to claim 3 wherein D is N.

8. A compound of claim 1 selected from:
(5Z)-2-[(2,6-dichlorophenyl)amino]-5-(pyrazolo[1,5-a]pyridin-5-ylmethylidene)-1,3-thiazol-4(5H)-one;
(5Z)-2-[(2,6-dichlorophenyl)amino]-5-(imidazo[1,2-a]pyridin-6-ylmethylidene)-1,3-thiazol-4(5H)-one;
methyl 5-{(Z)-[2-[(2,6-dichlorophenyl)amino]-4-oxo-1,3-thiazol-5(4H)-ylidene]methyl}pyrazolo[1,5-a]pyridine-3-carboxylate;
ethyl 6-{(Z)-[2-[(2,6-dichlorophenyl)amino]-4-oxo-1,3-thiazol-5(4H)-ylidene]methyl}imidazo[1,2-a]pyridine-3-carboxylate;
N-(4-chloro-3-{[(5Z)-5-(imidazo[1,2-a]pyridin-6-ylmethylidene)-4-oxo-4,5-dihydro-1,3-thiazol-2-yl]amino}phenyl)cyclobutanecarboxamide;
N-(4-chloro-3-{[(5Z)-4-oxo-5-(pyrazolo[1,5-a]pyridin-5-ylmethylidene)-4,5-dihydro-1,3-thiazol-2-yl]amino}phenyl)cyclobutanecarboxamide;
N-(4-chloro-3-{[(5Z)-4-oxo-5-(pyrazolo[1,5-a]pyridin-5-ylmethylidene)-4,5-dihydro-1,3-thiazol-2-yl]amino}phenyl)-2-methylpropanamide;
N-(4-chloro-3-{[(5Z)-5-(imidazo[1,2-a]pyridin-6-ylmethylidene)-4-oxo-4,5-dihydro-1,3-thiazol-2-yl]amino}phenyl)-2-methylpropanamide; and
N-(4-chloro-3-{[(5Z)-4-oxo-5-(pyrazolo[1,5-a]pyridin-5-ylmethylidene)-4,5-dihydro-1,3-thiazol-2-yl]amino}phenyl)-2,2-dimethylpropanamide;
and/or a pharmaceutically acceptable salt, solvate, hydrate or pro-drug thereof.

9. A pharmaceutical composition comprising a compound according to claim 1, and/or a pharmaceutically acceptable salt, hydrate, solvate or pro-drug thereof and a pharmaceutically acceptable carrier.

10. A process for preparing a pharmaceutical composition containing a pharmaceutically acceptable carrier or diluent and an effective amount of a compound of Formula I as described in claim 1 and/or a pharmaceutically acceptable salt, hydrate, solvate or pro-drug thereof, which process comprises bringing the compound of Formula I and/or a pharmaceutically acceptable salt, hydrate, solvate or pro-drug thereof into association with a pharmaceutically acceptable carrier or diluent.

11. A method of inhibiting YAK3 in a mammal; comprising, administering to the mammal a therapeutically effective amount of a compound of the Formula I, as described in claim 1, or a pharmaceutically acceptable salt, hydrate, solvate or pro-drug thereof.

12. A method of treating or preventing diseases of the erythroid and hematopoietic systems, caused by the YAK3 imbalance or inappropriate activity; comprising, administering to a mammal a therapeutically effective amount of a compound of claim 1, or a pharmaceutically acceptable salt, hydrate, solvate or pro-drug thereof and one or more of pharmaceutically acceptable carriers, diluents and excipients.

13. A method of claim 12 in which diseases of the erythroid and hematopoietic systems are selected from the group consisting of: anemia, aplastic anemia, myelodysplastic syndrome, myelosuppression, and cytopenia.

14. A method of treating or preventing diseases selected from the group consisting of: anemia, aplastic anemia, myelodysplastic syndrome, myelosuppression, and cytopenia; comprising, administering to a mammal a therapeutically effective amount of a compound of claim 1, or a pharmaceutically acceptable salt, hydrate, solvate or pro-drug thereof and one or more of pharmaceutically acceptable carriers, diluents and excipients.

15. The method of claim 12 wherein the mammal is a human.

16. A method of treating diseases of the hematopoietic system, in a mammal in need thereof, which comprises: administering to such mammal a therapeutically effective amount of
a) a compound of Formula I, as described in claim 1 and/or a pharmaceutically acceptable salt, hydrate, solvate or pro-drug thereof; and
b) EPO or a derivative thereof.
